(19) United States
(12) Plant Patent Application Publication
(10) Pub. No.: US 2009/0235398 P1
(43) Pub. Date: Sep. 17, 2009

(54) Kiwifruit variety named 'Y374'

(50) ;

(76) Inventor: Donald Alfred Skelton, Huntly (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 12/284,744

(22) Filed: Sep. 24, 2008

Related U.S. Application Data
(60) Provisional application No. 61/069,212, filed on Mar. 12, 2008.

(51) Int. Cl. A01H 5/00

(52) U.S. Cl. PLT/156

(57) ABSTRACT

A new and distinct kiwifruit variety is disclosed. The variety results from selection among a population of seedlings derived from crossing the kiwifruit selections known as ‘A17’ (not patented) and ‘R4’ (not patented). The fruit of the plant of this new kiwifruit variety is characterized by its internal medium-yellow color. The new kiwifruit variety has been named ‘Y374.’

### CROSS-REFERENCE TO RELATED APPLICATIONS

This application claims the benefit of U.S. Provisional Application No. 61/069,212, filed on Mar. 12, 2008, the entire contents of which are hereby incorporated by reference and should be considered a part of this specification.

### LATIN NAME OF THE GENUS AND SPECIES OF THE PLANT CLAIMED

Actinidia chinensis

### VARIETY DENOMINATION

‘Y374’

### BACKGROUND AND SUMMARY OF THE INVENTION

The invention relates to the discovery and asexual propagation of a new and distinct variety of kiwifruit, Actinidia chinensis cv. ‘Y374’, as herein described and illustrated. The new kiwifruit variety ‘Y374’ was selected from a population of seedlings derived from crossing two kiwifruit selections A17 (unpatented) and R4 (unpatented) using controlled pollination in the course of a planned kiwifruit variety breeding program. The cross was made in October 1999 by Donald Alfred Skelton at Rangiriri, New Zealand.

The new kiwifruit variety ‘Y374’ may be distinguished from presently available cultivars such as, for example, Actinidia chinensis variety ‘Hort16A’ (U.S. Plant Pat. No. 11,066), by the following distinguishing characteristics: the fruit of the new kiwifruit variety ‘Y374’ matures earlier than the fruit of ‘Hort16A’, and the fruit of the new kiwifruit variety ‘Y374’ expresses a medium-yellow flesh color.

The new kiwifruit variety ‘Y374’ may be distinguished from other presently available kiwifruit varieties featuring medium-yellow flesh fruit such as, for example, ‘Y363’ (unpatented), ‘D15’ (unpatented), ‘X273’ (unpatented), and ‘Skelton A19’ (unpatented), by the following distinguishing characteristics: the fruit shape of the new variety ‘Y374’ is obovoid compared with that of ‘Y363’, which is oblong. The fruit size of the new variety ‘Y374’ is large compared with that of‘D15’, which is medium, and ‘X273’, which is small. Furthermore, the beak of the new variety ‘Y374’ is very small compared with that of ‘Skelton A19’, which is apparent.

Asexual propagation of the new kiwifruit variety ‘Y374’ by grafting shows that the unique combination of characteristics of the variety come true to form and are established and transmitted through succeeding propagation. In order to obtain true-to-type clones of the initial plant, asexually propagated plants were obtained by grafting dormant buds from the original seedling onto rootstocks.

### BRIEF DESCRIPTION OF THE FIGURES

The accompanying photographs illustrate in full color typical specimens of the fruit, flowers, and leaves of the new variety cv. ‘Y374’. The colors as depicted are as nearly true as is reasonably possible in a color representation of this type.

FIG. 1 is a photograph of the fruit of the new kiwifruit variety ‘Y374’.

FIGS. 2A & 2B are photographs of the stem and stylar end views of fruit of the variety ‘Y374’.

FIG. 3 is a photograph of the fruit of the new kiwifruit variety ‘Y374’ fruit in longitudinal-section revealing the medium-yellow pigmentation of the fruit at maturity for consumption.

FIG. 4 is a photograph of the flowers of the new kiwifruit variety ‘Y374’.

FIG. 5 is a photograph of a mature leaf of the new kiwifruit variety ‘Y374’.

### DETAILED BOTANICAL DESCRIPTION OF THE INVENTION

The following is a detailed description of the new variety. The specimens described were grown at Rangiriri, New Zealand. The observations were made in the 2005 to 2008 seasons on vines grafted onto Chinensis and Deliciosa rootstocks, and managed under standard orchard practice. Random measurements of each characteristic were obtained from samples of 6 plants unless otherwise stated.

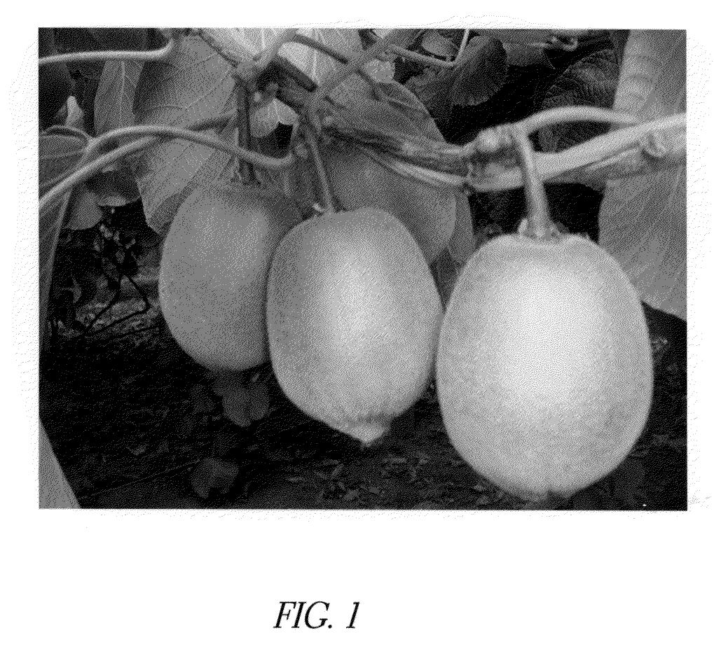

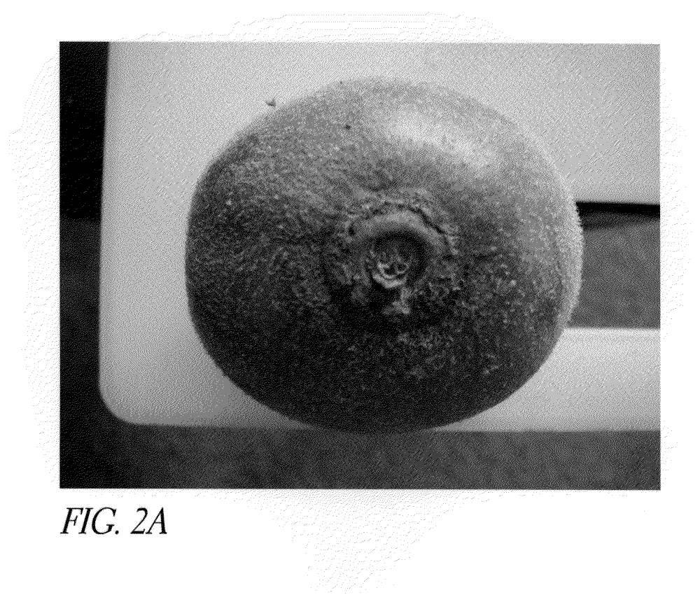

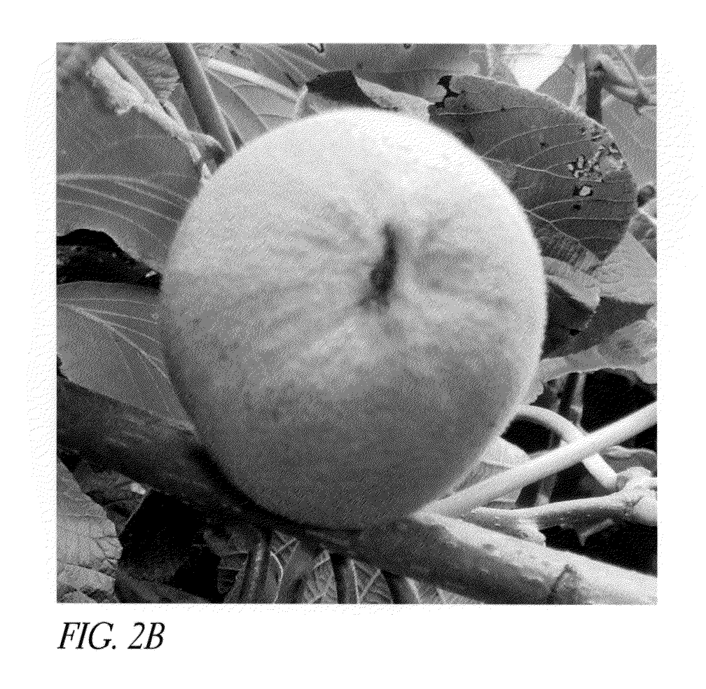

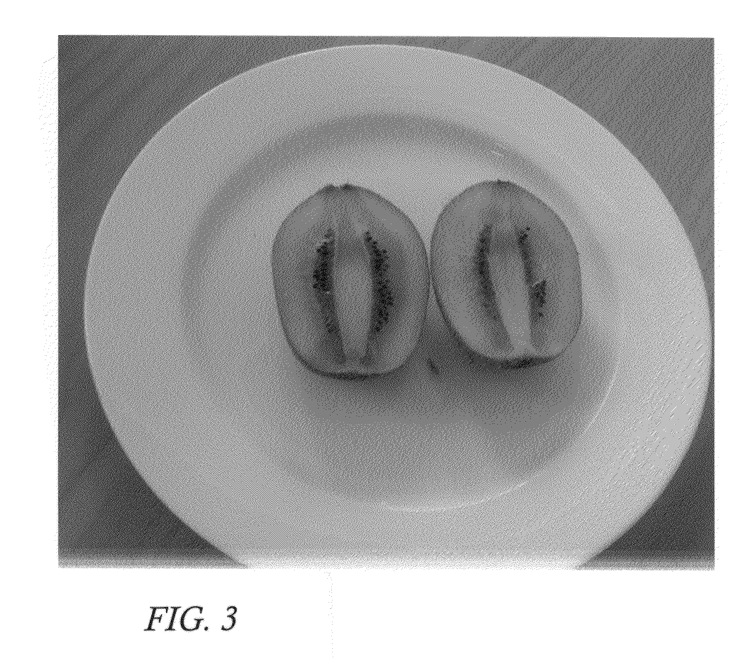

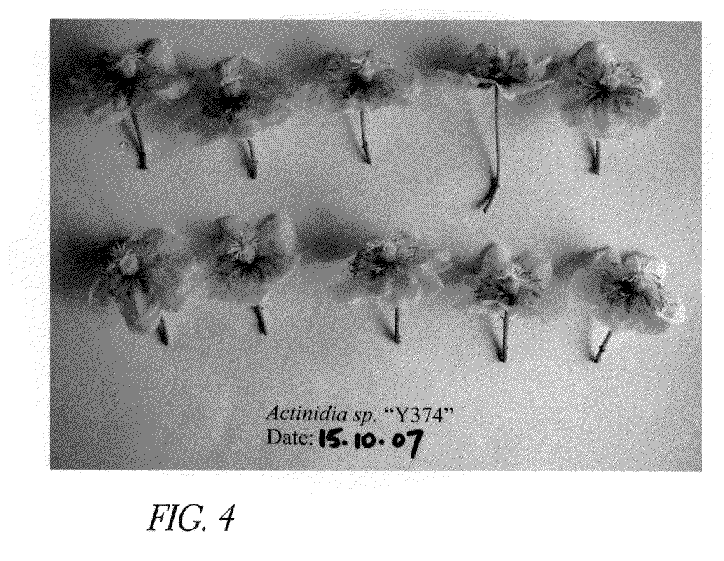

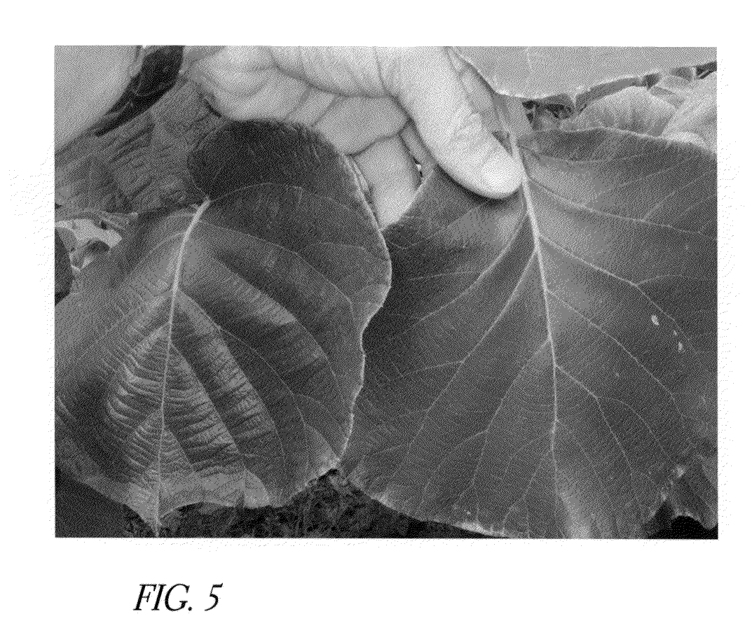

Horticultural terminology is used in accordance with UPOV guidelines for kiwi. All dimensions are in millimeters, and all weights are in grams (unless otherwise stated). Certain characteristics of this variety, such as growth and color, may change with changing environmental conditions (e.g., light, temperature, moisture), nutrient availability, rootstocks, or other factors. Color descriptions and other terminology are used in accordance with their ordinary dictionary descriptions, unless the context clearly indicates otherwise. Color names beginning with a capital letter designate values based upon the R.H.S. Colour Chart published by The Royal Horticultural Society, London, England.
Plant and foliage: The plant is a female plant that expresses a twining habit of medium vigor. Tomentose hairs are absent from the young shoot. Anthocyanin coloration of the growing tip is not present. The stem of the plant is smooth bark and colored Greyed-orange (R.H.S. 176B) on the upper side. Hairs are absent from the plant stem. The lenticels are medium in number. The size of the bud support is large. The leaf scar is deep. The mature leaf is broad ovate in shape with a mostly rounded leaf tip (FIG. 5) and typically averages approximately 210 mm in length and approximately 240 mm in width. The leaf bases are free. The petiole length averages approximately 80 mm. There is medium anthocyanin coloration on the upper side of the petiole. The color of the leaf blade is Green (R.H.S. 138A) on the upper surface and green (R.H.S. 138C) on the lower surface.
Inflorescence: The number of flowers in each inflorescence is typically between one to three flowers. The pedicel length averages approximately 37.4 mm. The sepals are Greyed-green (R.H.S. 194B) in color. The mean number of petals per flower is approximately 7. The petals are arranged overlapping, and are Yellow (R.H.S. 11C) when fully open.
Fruit: The fruit are overall large in size, averaging approximately 129 ǵ in weight. The fruit's dimensions average approximately 70 mm in length and approximately 65 mm maximum width, with a core diameter averaging approximately 13 mm in a first direction and 5 mm along a second direction perpendicular to the first direction. The general fruit shape in longitudinal section is obovoid, with the general shape of the stylar end being rounded (FIGS. 1 and 2). The fruit skin is absent of fruit hairs. The inner pericarp is colored Medium-yellow (R.H.S. 4C) at maturity for consumption. The fruit average approximately 16% Brix at maturity for consumption.
Cultivation: Vegetative bud break typically occurs in early September with flowering commencing in early October. The fruit attain maturity for harvest in approximately early March. All observations made under growing conditions at Ranigiriri, New Zealand.

We claim:

1. A new and distinct kiwifruit variety substantially as herein described and illustrated, characterized by its internal medium-yellow color and obovoid fruit shape.
